(19) United States
(12) Patent Application Publication
(10) Pub. No.: US 2026/0365615 A1
(43) Pub. Date: Nov. 19, 2026

(54) PROCESS FOR PREPARING AT LEAST ONE POLYISOCYANATE FROM CO2

(71) Applicant: BASF SE, Ludwigshafen am Rhein (DE)

(72) Inventors: Koenraad VANDEWALLE, Antwerp (BE); Holger FRIEDRICH, Ludwigshafen am Rhein (DE); Jens FERBITZ, Ludwigshafen am Rhein (DE); Torsten MATTKE, Ludwigshafen am Rhein (DE)

(21) Appl. No.: 19/138,724

(22) PCT Filed: Dec. 13, 2023
(86) PCT No.: PCT/EP2023/085675

§ 371 Date: Jun. 13, 2025

(30) Foreign Application Priority Data
Dec. 14, 2022 (EP) 22213402.5

(51) Int. Cl. C07C 265/14; C01B 3/06; C07C 1/12; C07C 1/20; C07C 15/04; C07C 15/06; C07C 29/15; C07C 68/08; C07C 69/96; C07C 201/08; C07C 205/06; C07C 209/36; C07C 263/10

(52) U.S. Cl. CPC C07C 265/14; C01B 3/06; C07C 1/12; C07C 1/20; C07C 15/04; C07C 15/06; C07C 29/15; C07C 68/08; C07C 69/96; C07C 201/08; C07C 205/06; C07C 209/36; C07C 263/10; C01B 2203/0283; C01B 2203/0415; C01B 2203/0475; C01B 2203/0872

(57) ABSTRACT

The present invention relates to a process for preparing polyisocyanates from CO₂ and a production unit for carrying out the process. Further, the present invention relates to a polyisocyanate and a polyisocyanate obtained according to the process of the present invention.

The present invention relates to a process for preparing polyisocyanates from CO₂ and a production unit for carrying out the process. Further, the present invention relates to a polyisocyanate and a polyisocyanate obtained according to the process of the present invention.

Di- and polyisocyanates are used in particular in the production of polyurethanes and coatings. Most widely used are aromatic di- and polyisocyanates such as TDI and PMDI. Besides, both aliphatic and cycloaliphatic di- and polyisocyanates are also known. Preparation thereof is usually done by phosgenation of the corresponding amines. Phosgene-free routes, for example via reductive or oxidative carbonylations, are also known, but play only a minor technical role. Preparation of phosgene is usually done from chlorine and CO in a heterogeneous catalytic gas phase reaction. In the phosgenation of the amines, HCl is formed as a by-product. In addition to its use in other processes (e.g. oxychlorination), processes for the recovery of chlorine from HCl are also known (electrolysis or Deacon process).

In the course of developing sustainable preparation processes, efforts are being made to use renewable raw materials, recycled materials or CO₂ itself. For example, WO2021/089737 discloses a process for the preparation of isocyanates by phosgenation of the corresponding amines, wherein the phosgene is produced from CO and chlorine and the CO is obtained from CO₂ by a reverse water gas shift (RWGS) reaction. The hydrogen required for this is provided by water electrolysis, whereby excess hydrogen can also be used in the production of the amines by hydrogenation of the nitro components.

WO2020/239716 discloses the pyrolysis of a polyurethane waste material in the course of which synthesis gas (CO/H₂), CO₂, low-molecular hydrocarbons and a residue are obtained. The CO₂ is converted in an electrolysis to CO and oxygen, whereby the oxygen is used for the combustion of the above-mentioned residue and possibly further amounts of polyurethane waste material. The resulting CO₂ is also fed to the above-mentioned CO₂ electrolysis. The resulting CO is used again in the synthesis of phosgene for the production of isocyanates. However, there is still a need to provide processes for the preparation of polyisocyanates which are more and more sustainable.

Therefore, it was an object of the present invention to provide an improved process for preparing polyisocyanates which exhibits good performance while showing improved sustainability compared to processes already known in the art. Indeed, it was an object of the present invention to provide a process for preparing polyisocyanates with reduced environmental impact while being cost effective.

Therefore, the present invention relates to a process for preparing at least one polyisocyanate R(—NCO)x from CO₂, the process comprises
(i) providing a stream S1 comprising CO₂;
(ii) providing a stream S2 comprising H₂;
(iii) passing and contacting a portion of S1 obtained according to (i) with a portion of S2 obtained according to (ii) into a reaction unit RURwGs for a reverse water gas shift reaction, obtaining a stream S3 comprising CO and H₂ and a stream SH comprising H₂O;
(iv) preparing at least one polyamine R(—NH 2)x, with x=2 or more, comprising
   (iv.1) passing a portion of S3 obtained according to (iii), or a portion of S1 and a portion of S2 obtained according to (i) and (ii), respectively, into an alcohol production unit PU1 and contacting CO and H₂ comprised in said portion of S3 into PU1, or contacting S1 and S2 in PU1, obtaining a stream SM comprising an alcohol having 1 to 4 carbon atoms; or
   (iv.1′)passing a portion of S1 and a portion of S2 obtained according to (i) and (ii), respectively, into an alkane production unit PU2 and contacting said portion of S1 with said portion of S2 into PU2, obtaining a stream SM′ comprising a C₁-C₄ alkane;
   (iv.2) passing a portion of SM obtained according to (iv.1), or a portion of SM′ obtained according to (iv.1′), in an aromatic production unit PU_{MTA} and reacting the alcohol having 1 to 4 carbon atoms comprised in SM, or the C₁-C₄ alkane comprised in SM′, in PU_{MTA}, obtaining a stream SA comprising at least one aromatic compound;
   (iv.3) reacting SA obtained according to (iv.2) with nitric acid in a nitration reaction unit RU_{N}, obtaining a stream SN comprising the corresponding at least one nitrated aromatic compound;
   (iv.4) passing SN obtained according to (iv.3) into an amine production unit APU and reacting the at least one nitrated aromatic compound comprised in SN in APU, obtaining a stream S4 comprising at least one polyamine R(—NH₂)x;
(v) preparing phosgene, comprising
   (v.1) providing a stream S5 comprising Cl₂;
   (v.2) passing a portion of S3 into a purification unit, obtaining a stream S_{CO} comprising CO and a stream S_{CH} depleted in CO compared to the portion of S3, comprising H₂;
   (v.3) passing at least a portion of S_{CO} obtained according to (v.2), into a reaction unit for preparing phosgene R_{UP} and contacting said at least portion of S_{CO} with S5 obtained according to (v.1) into R_{UP}, obtaining a stream S6 comprising phosgene;
(vi) passing S4 obtained according to (iv.4) comprising at least one polyamine R(—NH₂)x into a reaction unit RU and reacting S4 with phosgene comprised in S6 obtained according to (v.3), obtaining a stream SP comprising the at least one polyisocyanate R(—NCO)x.

In the context of the present invention, CO₂ provided in (i) can obtained from any processes known to the skilled person. For example, at least a portion of CO₂ provided in (i) as S1 can be obtained from combustion of methane and/or obtained from recycling of waste materials.

In this regard, the waste materials may comprise one or more of polyurethane, polyurethane urea, polyethylene, polypropylene, polystyrene, polyamine, for example the waste materials comprises polyurethane. For example, at least a portion of CO₂ comprised in S1 can be obtained from pyrolysis of waste materials, such as waste polyurethane and/or waste polyurethane urea.

Preferably, at least a portion of CO₂ comprised in S1 is obtained from hydrolysis, hydroglycoly-sis, hydroaminolysis or hydroammonolysis of one or more of polyurethane and polyurethane urea.

Preferably, the reverse water gas shift reaction performed in RURwGs according to (iii) is performed at a temperature in the range of from 500 to 1200 °C, more preferably in the range of from 700 to 1100 °C, more preferably in the range of from 800 to 1000 °C, more preferably in the range of from 850 to 1000 °C, more preferably in the range of from 900 to 1000 °C

Preferably, the reverse water gas shift reaction performed in RU_{RWGS} according to (iii) is performed at a pressure in the range of from 0.5 to 20 bar(abs), more preferably in the range of from 2 to 18 bar(abs), more preferably in the range of from 2 to 15 bar(abs).

Preferably, the molar ratio of H₂ to CO₂ for the reverse water gas shift reaction performed in RU_{RWGS} according to (iii) is in the range of from 0.5:1 to 10:1, more preferably in the range of from 0.8:1 to 5:1, more preferably in the range of from 1:1 to 3:1, more preferably in the range of from 1.5:1 to 3:1, more preferably in the range of from 2:1 to 3:1.

The reverse water gas shift reaction are preferably performed according to known processes in the art such as for example those defined in E. Rezaei, S. Dzuryk “Techno-economic comparison of reverse water gas shift reaction to steam and dry methane reforming reactions for syngas production”, Chemical Engineering Research and Design, Vol 144(2019 ), S. 354-369, EP2175986, CN103183346 and U.S. Pat. No. 8,946,308.

Preferably, (iii) comprises
(iii.1) passing and contacting a portion of S1 obtained according to (i) with a portion of S2 obtained according to (ii) into a reaction unit RU_{RWGS} for a reverse water gas shift reaction, obtaining a stream S13 comprising CO, CO₂, H₂ and H₂O;
(iii.2) cooling the stream S13 obtained according to (iii.1), in a condensation unit CU, obtaining a gaseous stream S3, depleted in water, compared to S13, comprising CO and H₂, and a liquid stream SH comprising H₂O; or
(iii.2′) comprises
   (iii.2′.1) cooling the stream S13 obtained according to (iii.1), in a condensation unit CU, obtaining a gaseous stream S23, depleted in water, compared to S13, comprising CO and H₂, and a liquid stream SH comprising H₂O;
   (iii.2′.2) passing S23 obtained according to (iii.2′.1) into an amine scrubbing unit for removing CO₂, obtaining a gaseous stream S3, depleted in CO₂, compared to S23, comprising CO and H₂.

Preferably, (iii) comprises
(iii.1) passing and contacting at least a portion of S1 obtained according to (i) with at least a portion of S2 obtained according to (ii) into a reaction unit RU_{RWGS} for a reverse water gas shift reaction, obtaining a stream S13 comprising CO, CO₂, H₂ and H₂O;
(iii.2) cooling the stream S13 obtained according to (iii.1), in a condensation unit CU, obtaining a gaseous stream S3, depleted in water, compared to S13, comprising CO and H₂, and a liquid stream SH comprising H₂O.

Alternatively, preferably (iii) comprises
(iii.1) passing and contacting at least a portion of S1 obtained according to (i) with at least a portion of S2 obtained according to (ii) into a reaction unit RURwGs for a reverse water gas shift reaction, obtaining a stream S13 comprising CO, CO₂, H₂ and H₂O;
(iii.2′) comprises
   (iii.2′.1) cooling the stream S13 obtained according to (iii.1), in a condensation unit CU, obtaining a gaseous stream S23, depleted in water, compared to S13, comprising CO and H₂, and a liquid stream SH comprising H₂O;
   (iii.2′.2) passing S23 obtained according to (iii.2′.1) into an amine scrubbing unit for removing CO₂, obtaining a gaseous stream S3, depleted in CO₂, compared to S23, comprising CO and H₂.

Preferably, the condensation unit comprises a condensation mean and one or more heat exchangers suitable for cooling down and condensation, such as plate, shell-and-tube and air cooler.

Preferably, the removal of CO₂ in the amine scrubbing unit is performed in presence of any suitable material to selectively chemisorb CO₂. Preferably, such material is one or more of NaOH and amines, such as monoethanolamine (MEA), diethanolamine (DEA) or methyl diethanolamine (MDEA).

Preferably, the amine scrubbing unit comprises absorption and desorption column.

Preferably, at least a portion of the CO₂, more preferably the CO₂, removed from S13 or S23 by the amine scrubbing unit is recycled as a portion of S1.

Preferably, the weight ratio of CO to H₂ in S3 is in the range of from 1:1 to 30:1, preferably in the range of from 2:1 to 20:1, more preferably in the range of from 5:1 to 15:1.

### Preferably from 25 to 100 weight-% of S3 consist of CO and H₂.

More preferably from 85 to 100 weight-%, more preferably from 90 to 100 weight-%, more preferably from 95 to 100 weight-%, more preferably from 98 to 100 weight-%, of S3 consists of CO and H₂, when S3 is obtained from (iii.2′.2).

Alternatively, more preferably from 30 to 95 weight-%, more preferably from 40 to 85 weight-%, of S3 consist of CO and H₂, when S3 is obtained from (iii.2).

Preferably, according to (iv.1), from 75 to 95 weight-%, preferably from 85 to 93 weight-%, of S3 is passed in the alcohol production unit PU1.

Preferably, PU1 comprises an alcohol production reactor, wherein (iv.1) comprises passing a portion of S3 into PU1 and contacting CO and H₂ comprised in said portion of S3 with a catalyst in the alcohol production reactor at a temperature in the range of from 150 to 500 °C, more preferably in the range of from 180 to 320 °C, more preferably in the range of from 200 to 300 °C, more preferably in the range of from 205 to 280 °C, obtaining a stream SM comprising an alcohol having 1 to 4 carbon atoms.

Preferably, contacting CO and H₂ with a catalyst in the alcohol production reactor according to (iv.1) is performed at a pressure of up to 380 bar(abs), more preferably up to 300 bar(abs), more preferably in the range of from 30 to 120 bar(abs), more preferably in the range of from 50 to 100 bar(abs).

Preferably, the catalyst comprised in the alcohol production reactor comprises at least copper, zinc oxide and alumina.

In the context of the present invention, the alcohol having 1 to 4 atoms is selected from the group consisting of ethanol, methanol, propanol and butanol.

Preferably, in SM, the alcohol having 1 to 4 atoms is methanol or ethanol, more preferably is methanol.

Preferably, the stream SM is further purified, more preferably by passing said stream in a purification unit. Such processes are well known in the art and disclosed in the documents listed be-low.

Preferably, methanol is produced in (iv.1) from a portion of S3 according to methods known in the art such as those disclosed in DE69808983, EP3901126, EP3441381, EP0336378, EP2100869, EP2281792, EP0128400, and J. Ott et. al. “Methanol” in “Ullmann's Encyclopedia of industrial chemistry”, Wiley-VCH Verlag Gmbh & Co KGaA, Weinheim, 2012.

Alternatively, preferably methanol is produced in (iv.1) from a portion of S1 and a portion of S2 according to methods known in the art such as those disclosed in Sofiane Arab: “Development of a process for methanol synthesis from CO₂”. PhD, Université Nancy, 2014, Laetitia Angelo “Development of catalysts for the carbon dioxide hydrogenation into methanol”, PhD Laboratoire des matériaux, surfaces et procédés pour la catalyse, 2014, EP 0011150 and Section 5.3.1 in J. Ott et. al. “Methanol” in “Ullmann's Encyclopedia of industrial chemistry”, Wiley-VCH Verlag GmbH & Co KGaA, Weinheim, 2016.

Preferably, PU2 comprises an alkane production reactor, wherein (iv.1′) comprises passing a portion of S1 and a portion of S2 into PU2 and contacting CO₂ comprised in said portion of S1 and H₂ comprised in said portion of S2 in the alkane production reactor PU2 at a temperature in the range of from 250 to 600 °C, more preferably in the range of from 280 to 500 °C, more preferably in the range of from 300 to 400 °C, obtaining a stream SM′ comprising a C₁-C₄ alkane.

Preferably, the weight ratio of CO₂ to H₂ in (iv.1′) is in the range of from 1:1 to 1:20, preferably in the range of from 1:1,5 to 1:10, more preferably in the range of from 1:2 to 1:5.

In the context of the present invention, C₁-C₄ alkane is selected from the group consisting of ethane, methane, propane and butane.

Preferably, in SM′, the C₁-C₄ alkane is methane or ethane, more preferably methane.

Preferably, the stream SM′ comprising a C₁-C₄ alkane is obtained via the Sabatier reaction.

Preferably, (iv.2) comprises
passing and contacting a portion of SM obtained according to (iv.1) or a portion of SM′ obtained according to (iv.1′) with a catalyst, in an aromatic production unit PU_{MTA}, obtaining a stream SA comprising at least one aromatic compound, wherein the aromatic compound is selected from the group consisting of benzene, toluene and xylene, more preferably benzene or toluene.

Preferably, PU_{MTA} comprises at least one aromatic conversion reactor, preferably any reactor suitable for heterogeneous gas phase reactions, such as a fixed bed reactor and fluidized bed reactor.

Preferably, when (iv.1) is performed, (iv.2) comprises
(iv.2.1) passing a portion of SM obtained according to (iv.1) in the at least one aromatic conversion reactor, more preferably at least one methanol-to-aromatics conversion reactor;
(iv.2.2) contacting the stream SM with a catalyst in the at least one reactor, more preferably at a temperature in the range of from 250 to 750 °C, more preferably in the range of from 300 to 600 °C, obtaining a stream SA1 comprising water and a mixture of aromatic compounds, the aromatic compounds being benzene, toluene and xylene;
(iv.2.3) passing the stream SA1 obtained according to (iv.2.2) in a separation unit SU, obtaining a stream SA comprising benzene or toluene.

Optionally, SA1 further comprises C₁-C₄ alkane, more preferably methane or ethane. Preferably, the process further comprises passing said alkane, after separation from the aromatic compounds obtained according to (iv.2.2), in a second aromatic conversion unit, obtaining aromatic compounds.

Preferably, the pressure in the reactor in (iv.2.2) is in the range of from 0.5 to 30 bar(abs), more preferably in the range of from 0.75 to 10 bar(abs), more preferably in the range of from 0.8 to 6 bar(abs).

Preferably, the separation unit SU used in (iv.2.3) is one or more of a distillation column, ad-sorption column and phase separation.

Optionally, the process further comprises
demethylation of xylene separated from SA1, obtaining benzene or toluene. Such process is known to the skilled person as disclosed for example in U.S. Pat. No. 2,786,876.

Preferably, the stream SA comprising at least one aromatic compound is obtained by processes well-known in the art such as those disclosed in EP0090284, CN104496743, CN104549481, EP 2188234, W. O. Haag, R. M. Lago, and P. G. Rodewald, J. Mol. Catal. 17, 161(1982 ), R. Le Van Mao, P. Levesque, B. Sjariel, and D. T. Nguyen, Can. J. Chem. Eng.64,462 (1986) and G. Pop and co-workers, Ind. Eng. Chem. Prod. Res. Dev.25, 208-213 (1986); or such as those disclosed in U.S. Pat. No. 11,084,764, EP2595943, US2011060176 and Ismagilov, Z. R., et al., “Direct Conversion of Methane on Mo/ZSM-5 Catalysts to Produce Benzene and Hydrogen: Achievements and Perspectives,” Energy & Environmental Science 1, 5(2008 ), 526-541.

Preferably, when (iv.1′) is performed, (iv.2) comprises
(iv.2.1′) passing a portion of SM′ obtained according to (iv.1′) in the at least one aromatic conversion reactor, more preferably at least one methane-to-aromatics conversion reactor;
(iv.2.2′) contacting the stream SM′ with a catalyst in the reactor, more preferably at a temperature in the range of from 600 to 1000 °C, more preferably in the range of from 625 to 900 °C, more preferably in the range of from 650 to 850 °C, obtaining a stream SA1′ comprising a mixture of aromatic compounds, the aromatic compounds being benzene, toluene and xylene;
(iv.2.3′) passing the stream SA1′ obtained according to (iv.2.2′) in a separation unit SU′, obtaining a stream SA comprising benzene or toluene.

Preferably, (iv.2.2′) is performed at a pressure in the range of from 1 to 20 bar(abs), more preferably in the range of from 2 to 10 bar(abs), more preferably in the range of from 2 to 6 bar(abs).

Preferably, (iv.3) comprises
reacting SA obtained according to (iv.2) with nitric acid in a nitration reaction unit RU_{N}, obtaining a stream SN comprising the corresponding at least one nitrated aromatic compound, wherein more preferably the at least one nitrated aromatic compound is selected from the group consisting of nitrobenzene and dinitrotoluene.

Preferably, (iv.3) comprises reacting SA comprising benzene obtained according to (iv.2) with nitric acid, more preferably with a mixture of nitric acid and sulfuric acid, in a nitration reaction unit RU_{N}, obtaining a stream SN comprising nitrobenzene.

Preferably, the nitration reaction running in RU_{N} for preparing nitrobenzene is an adiabatic reaction, more preferably at a temperature in the range of from 50 to 150 °C

Preferably, the nitration reaction runs in run for preparing nitrobenzene with an excess of benzene.

Preferably, the nitration reaction running in RU_{N} for preparing dinitroluene is performed in two steps, in a first step, the nitration reaction is an isothermal or adiabatic reaction to obtain mono-nitrotoluene, and in a second step, the nitration reaction is an isothermal reaction to obtain dinitrotoluene.

Preferably, the nitration reaction runs in RU_{N} for preparing dinitroluene at a temperature in the range of from 30 to 120 °C.

For example, the nitration can be performed by processes known in the art such that those disclosed in G. Booth “Nitro compounds, Aromatic” Vol. 24 in “Ullmann's Encyclopedia of industrial chemistry”, Wiley-VCH Verlag Gmbh & Co KGaA, Weinheim, 2012, H. Hermanns et. al. “Chapter 21-Industrial nitration of toluene to dinitrotoluene” in L. F. Albright (ed.) and “Nitration-Recent Laboratory and Industrial Developments”, ACS Symposium Series 623, American Chemical Society, Washington, DC 1996.

Preferably, (iv.4) comprises
passing SN obtained according to (iv.3) in an amine production unit APU being a hydrogenation unit and contacting the at least one nitrated aromatic compound comprised in SN with H₂ into the hydrogenation unit, obtaining a stream S4 comprising at least one corresponding aromatic polyamine R(—NH₂)x.

Preferably, the at least one polyamine R(—NH₂)x is selected from the group consisting of monomeric methylene diphenylene diamine, polymethylene polyphenylene polyamine, a mixture of methylene diphenylene diamine and polymethylene polyphenylene polyamine, toluenediamine (TDA), isomers of xylylenediamine (XDA), isomers of diaminobenzene, 2,6-xylidine, naph-thylene-1,5-diamine (1,5-NDA), 1,4-diaminobutane, 1,5-diaminopentane (PDA), 1,6-diaminohexane (HDA), 1,8-diaminooctane, 1,9-diaminononane, 1,10-diaminodecane, 2,2-dimethyl-1,5-diaminopentane, 2-methyl-1,5-pentanediamine (MPDA), 2,4,4(or 2,2,4)-trimethyl-1,6-diaminohexane (TMDA), 1,3-and 1,4-diaminocyclohexane, 1-amino-3,3,5-trimethyl-5-aminomethylcyclohexane (IPDA), 2,4-or 2,6-diamino-1-methylcyclohexane (H 6-TDA), 1-amino-1-methyl-4(3)-aminomethylcyclohexane (AMCA), 1,3-bis(aminomethyl)cyclohexane, 1,4-bis(ami-nomethyl)cyclohexane, bis(aminomethyl)norbornane (NBDA), 4,4′-diaminodicyclohexylme-thane, 2,4′-diaminodicyclohexylmethane, triaminocyclohexane, tris(aminomethyl)cyclohexane, triaminomethylcyclohexane, 1,8-diamino-4-(aminomethyl)octane, undecane- 1,6,11-triamine, 1,7-diamino-4-(3-aminopropyl)heptane, 1,6-diamino-3-(aminomethyl)hexane and 1,3,5-tris(aminomethyl)cyclohexane. It is more preferred that the at least one polyamine R(—NH₂)x is selected from the group consisting of monomeric methylene diphenylene diamine, polymethylene polyphenylene polyamine, a mixture of monomeric methylene diphenylene diamine and polymethylene polyphenylene polyamine, and toluenediamine (TDA).

It is preferred that the at least one polyamine R(—NH₂)x is toluenediamine (TDA), preferably TDA comprises a mixture of 2,4-TDA (80 wt.-%) and 2′6-TDA (20 wt.-%).

Preferably, (iv.4) comprises
(iv.4.1) passing SN comprising dinitrotoluene obtained according to (iv.3) into a hydrogenation unit HU under hydrogenation conditions, obtaining a stream S4 comprising toluenediamine (TDA).

Preferably, the hydrogenation unit used in (iv.4.1) is a three-phase reactor.

Alternatively, it is preferred that the at least one polyamine R(—NH₂)x is selected from the group consisting of monomeric methylene diphenylene diamine, polymethylene polyphenylene polyamine, and a mixture of monomeric methylene diphenylene diamine and polymethylene polyphenylene polyamine, more preferably selected from the group consisting of monomeric methylene diphenylene diamine and a mixture of monomeric methylene diphenylene diamine (mMDA) and polymethylene polyphenylene polyamine (PMDA).

Preferably the monomeric methylene diphenylene diamine comprises, more preferably consists of, one or more 4,4′-diaminodiphenylmethane, 2,2′-diaminodiphenylmethane and 2,4′-diaminodiphenylmethane. It is more preferred that the at least one polyamine R(—NH₂)x is 4,4′-diaminodiphenylmethane.

In the context of the present invention, when the term “diamino diphenylmethane” or “MDA” is used, it encompasses all isomers, monomeric and polymethylene polyphenylene polyamine.

According to said alternative, preferably (iv.4) comprises
(iv.4.1′) preparing an aqueous stream SF comprising formaldehyde;
(iv.4.2′) passing SN obtained according to (iv.3) into a hydrogenation unit comprised in an amine production unit APU under hydrogenation conditions, obtaining a stream S14 comprising aniline;
(iv.4.3′) bringing in contact S14 obtained according to (iv.4.2′) with SF obtained according to
(iv.4.1′) in a condensation reactor R_{C} comprised in APU in presence of a catalyst, more preferably an acidic catalyst, more preferably the acidic catalyst being hydrochloric acid, obtaining a stream S4 comprising one or more of monomeric methylene diphenylene diisocyanate and polymethylene polyphenylene polyisocyanate.

Preferably, (iv.4.2′) comprises passing SN obtained according to (iv.3) into a hydrogenation unit comprised in an amine production unit APU and contacting SN with a catalyst in said hydrogenation unit in the gas phase, obtaining a stream S14 comprising aniline.

It is also conceivable that the catalyst used in (iv.4.3′) is a heterogeneous catalyst.

Preferably, the process further comprises passing S14 into a purification unit, more preferably one or more distillation columns, more preferably at least two distillation columns, obtaining a purified stream S14 comprising aniline.

Preferably, the aniline is obtained by processes known in the art such as those disclosed in T. Kahl et. al. “Aniline” Vol.3 in “Ullmanns' Encyclopedia of industrial chemistry”, Wiley-VCH Verlag GmbH & Co KGaA, Weinheim, 2012.

Preferably, the hydrogenation unit used in (iv.4.2′) is a fluidized bed reactor.

Preferably, (iv.4.1′) comprises passing a portion of SM obtained according to (iv.1) in a reactor for preparing formaldehyde in presence of H₂O and O₂, obtaining an aqueous stream SF comprising formaldehyde.

Preparing formaldehyde is preferably performed according to processes known in the art such as those disclosed in A.W. Franz et. al. “Formaldehyde” in “Ullmanns'Encyclopedia of industrial chemistry”, Wiley-VCH Verlag Gmbh & Co KGaA, Weinheim, 2016.

Preferably, (v.1) comprises contacting and reacting a stream comprising HCl with a stream comprising O₂ in presence of a catalyst, obtaining a stream S5 comprising Cl₂.

For preparing Cl₂ according to the present invention processes well-known in the art can be used. For example, Cl₂ can be prepared according to the Deacon process as illustrated and disclosed in WO 2007/134771A1, WO 2011/111351A1, WO 2013/004651A1, WO 2013/060628 A 1, U.S. Pat. No. 2,418,930, WO 2012/025483, WO 2007137685, or via HCl electrolysis disclosed for example in U.S. Pat. No. 6,022,634, WO2003/31690, WO1999031297, EP1103636 and U.S. Pat. No. 3,236,760, or via NaCl electrolysis such as in EP0004903.

Preferably, the purification unit according to (v.2) is a CO—H₂ separation cold box CB.

Preferably, the process further comprises recycling at least a portion of S_{CH} obtained according to (v.2) in R_{RWGS}.

Preferably, when (iv.1) is performed, from 5 to 25 weight-%, more preferably from 7 to 20 weight-%, of S3 is fed into a purification unit according to (v.2), more preferably in a CO—H₂ separation cold box.

Alternatively, preferably, when (iv.1′) is performed, from 95 to 100 weight-%, more preferably from 98 to 100 weight-%, more preferably from 99 to 100 weight-%, of S3 is fed into a purification unit according to (v.2), more preferably in a CO—H₂ separation cold box CB.

Preferably, (v.2) comprises
passing a portion of S3 obtained according to (iii), more preferably (iii.2′), in a CO—H₂ separation cold box, obtaining a stream S_{CO} comprising CO and a stream S_{CH} depleted in CO compared to the portion of S3, comprising H₂.

Preferably from 99 to 100 weight-%, more preferably from 99.9 to 100 weight-%, more preferably from 99.99 to 100 weight-% of S_{CO} consists of CO.

Preferably, S_{CO} is substantially free of H₂, more preferably free of H₂. In other words, preferably at most 0.1 weight-% of S_{CO} consists of H 2, more preferably at most 0.01 weight-% of Sco consists of H 2, more preferably at most 0.001 weight-% of S_{CO} consists of H₂.

Preferably, (v.3) comprises
passing a portion of S3 obtained according to (iii), more preferably (iii.2′), or S_{CO} obtained according to (v.2), more preferably S_{CO} obtained according to (v.2), into a reaction unit for preparing phosgene RU_{P} and contacting said portion of S3 with S5 obtained according to (v.1) into RU_{P} in the presence of a catalyst, more preferably activated carbon, obtaining a stream S6 comprising phosgene.

Preferably, RU_{P} is a tube bundle reactor.

Preferably, the preparation of phosgene is a heterogeneous gas phase catalysis on activated carbon.

Phosgene is preferably prepared by processes know in the art such as those disclosed in Ullmann's Encyclopedia of industrial chemistry, Chapter “Phosgene” 5^{th} Ed., Vol. A 19, p 413 ff., VCH Verlagsgesellschaft mbH, Weinheim, 1991, and C. Ryan et. al. “Phosgene and related carbonyl halides”, Elsevier Science, 1996, ISBN 978-0-08-053880-8.

Preferably, (vi) comprises
(vi.1) introducing S4 obtained according to (iv.4) comprising at least one polyamine R(—NH₂)x and S6 obtained according to (v), and optionally a solvent, into a mixing device comprised in the reaction unit RU, more preferably a mixing nozzle, wherein the solvent is an aromatic solvent, more preferably an halogenated aromatic solvent, more preferably monochlorobenzene or dichlorobenzene, obtaining a reaction mixture;
(vi.2) passing the reaction mixture obtained according to (vi.1) in one or more reactors comprised in RU, obtaining a stream SP comprising the at least one polyisocyanate R(—NCO)x.

Preferably, the process further comprises
passing SP into a work-up unit, preferably a distillation unit, for removing the solvent if any.

Preferably the stream SP comprises the at least one polyisocyanate R(—NCO)x being selected from the group consisting of monomeric methylene diphenylene diisocyanate, polymethylene polyphenylene polyisocyanate, a mixture of monomeric methylene diphenylene diisocyanate and polymethylene polyphenylene polyisocyanate, toluene diisocyanate (TDI), isomers of xylylene diisocyanate (XDI), isomers of diisocyanatobenzene, xylene 2,6-isocyanate, naphthylene 1,5-diisocyanate (1,5-NDI), 1,4-diisocyanate, pentane 1,5-diisocyanate (PDI), hexane 1,6-diisocyanate (HDI), octane 1,8-diisocyanate, nonane 1,9-diisocyanate, decane, 1,10-diisocyanate, 2,2-dimethylpentane 1,5-diisocyanate, 2-methylpentane 1,5-diisocyanate (MPDI), 2,4,4(or 2,2,4)-trimethylhexane 1,6-diisocyanate (TMDI), cyclohexane 1,3-and 1,4-diisocyanate, 1-iso-cyanato-3,3,5-trimethyl-5-isocyanatomethylcyclohexane (IPDI), 2,4-or 2,6-diisocyanato-1-methylcyclohexane (H6-TDI), 1-isocyanato-1-methyl-4(3)-isocyanatomethylcyclohexane (AMCI), 1,3-bis(isocyanatomethyl)cyclohexane, 1,4-bis(isocyanatomethyl)cyclohexane, bis(isocy-anatomethyl)norbornane (NBDI), 4,4′-diisocyanatodicyclohexylmethane, 2,4′-diisocyanatodicyclohexylmethane, triisocyanatocyclohexane, tris(isocyanatomethyl)cyclohexane, triisocy-anatomethylcyclohexane, 1,8-diisocyanato-4-(isocyanatomethyl)octane, undecane 1,6,11-triiso-cyanate, 1,7-diisocyanato-4-(3-isocyanatopropyl)heptane, 1,6-diisocyanato-3-(isocyanatomethyl)hexane and 1,3,5-tris(isocyanatomethyl)cyclohexane, more preferably selected from the group consisting of monomeric methylene diphenylene diisocyanate, polymethylene polyphenylene polyisocyanate, a mixture of monomeric methylene diphenylene diisocyanate and polymethylene polyphenylene polyisocyanate, toluene diisocyanate (TDI) and isomers of xylylene diisocyanate (XDI), more preferably selected from the group consisting of monomeric methylene diphenylene diisocyanate, polymethylene polyphenylene polyisocyanate, a mixture of monomeric methylene diphenylene diisocyanate and polymethylene polyphenylene polyisocyanate and toluene diisocyanate (TDI).

Preferably, in SP, the at least one polyisocyanate R(—NCO)x is TDI, more preferably a mixture of 2,4-TDI and 2,6-TDI.

Preferably, TDIs a mixture of 2,4-TDI (80 weight-%) and 2,6-TDI (20 weight-%).

Preferably, in SP, the at least one polyisocyanate R(—NCO)x is selected from the group consisting of monomeric methylene diphenylene diisocyanate, polymethylene polyphenylene polyisocyanate and a mixture of monomeric methylene diphenylene diisocyanate and polymethylene polyphenylene polyisocyanate.

Preferably the monomeric methylene diphenylene diisocyanate (mMDI) comprises, more preferably consists of, one or more 4,4′-methylene(diphenyl diisocyanate) (4,4′-MDI), 2,2′-methylene (diphenyl diisocyanate) (2,2′-MDI) and 2,4′-methylene (diphenyl diisocyanate) (2,4′-MDI), more preferably 4,4′-methylene(diphenyl diisocyanate) (4,4′-MDI).

Preferably, the process further comprises separating monomeric methylene diphenylene diisocyanate and polymethylene polyphenylene polyisocyanate comprised in SP, by one or more of a distillation step and a crystallization step, obtaining one or more mixtures M1 of 2,4′-and 4,4′-MDI and a mixture M2 of monomeric methylene diphenylene diisocyanate and polymethylene polyphenylene polyisocyanate.

Preferably at least 22.5 %, more preferably at least 30 %, more preferably at least 50 % of the carbon atoms in the at least one polyisocyanate R(—NCO)x originates from CO₂.

The present invention further relates to a production unit for carrying out the process according to the present invention, the production unit comprising:
a reaction unit R_{RWGS} for a reverse water gas shift reaction for obtaining S3 comprising CO and H₂;
a means for introducing CO₂ in R_{RWGS};
a means for introducing H₂ in R_{RWGS};
a means for removing S3 from R_{RWGS};
an alcohol production unit PU1 for obtaining SM or an alkane production unit PU2 for obtaining SM′;
a means for passing a portion of S3 in PU1;
a means for passing a portion of S1 in PU2;
a means for removing SM from PU1or a means for removing SM′ from PU2;
an aromatic production unit PU_{MTA} for obtaining a stream SA comprising at least one aromatic compound;
a means for passing SM or SM′ into PU_{MTA};
a nitration reaction unit RU_{N} for obtaining a stream SN comprising at least one nitrated aromatic compound;
a means for removing SN from RU_{N};
an amine production unit APU for obtaining a stream S4 comprising at least one polyamine R(—NH₂)x, with x=2 or more;
a means for passing SN into APU;
a reaction unit R_{UP} for preparing phosgene as stream S6;
a means for passing S5 in RU_{P};
a purification unit, preferably a CO—H₂ separation cold box CB, for obtaining a stream Sco;
a means for passing S_{CO} into RU_{P};
a means for removing S_{CO} from the purification unit;
a means for removing S_{CH} from the purification unit;
a means for passing a portion of S3 into the purification or R_{UP};
a means for removing S6 from R_{UP};
a reaction unit RU for obtaining a stream SP comprising at least one corresponding polyisocyanate R(—NCO)x;
a means for passing S4 into RU;
a means for passing S6 into RU;
a means for removing SP from RU.

Preferably, the amine production unit APU comprises one or more of a hydrogenation unit and a condensation reaction unit.

Preferably, APU comprises a hydrogenation unit. Alternatively, APU comprises a hydrogenation unit and a condensation unit.

Preferably, the production unit further comprises a reactor for preparing formaldehyde from a portion of SM.

Preferably, the production unit further comprises one or more purification units, such as distillation column(s), flash drum(s), liquid-liquid separation unit(s), solid-liquid separation unit(s), such as filter, sieve, evaporator(s), dryer(s), crystallization unit(s) and extraction unit(s).

### Preferably, the Production Unit Further Comprises One or More Heat Exchangers.

The present invention further relates to a polyisocyanate R(—NCO)x characterized in that at least at least 22.5%, preferably at least 30%, more preferably at least 50% of its carbon atoms are obtained from CO₂. Preferably, the polyisocyanate R(—NCO)x is MDI. Alternatively, preferably, the polyisocyanate R(—NCO)x is TDI.

The present invention further relates to a polyisocyanate R(—NCO)x obtainable or obtained by a process according to any one of embodiments 1 to 28, wherein at least at least 22.5%, preferably at least 30%, more preferably at least 50% of its carbon atoms are obtained from CO₂. Preferably, the polyisocyanate R(—NCO)x is MDI. Alternatively, preferably, the polyisocyanate R(—NCO)x is TDI.

The present invention further relates to a use of the polyisocyanate R(—NCO)x according to the present invention for preparing polyurethane or polyurethane urea.

The present invention is further illustrated by the following set of embodiments and combinations of embodiments resulting from the dependencies and back-references as indicated. In particular, it is noted that in each instance where a range of embodiments is mentioned, for example in the context of a term such as “The process of any one of embodiments 1 to 3”, every embodiment in this range is meant to be explicitly disclosed for the skilled person, i.e. the wording of this term is to be understood by the skilled person as being synonymous to “The process of any one of embodiments 1, 2 and 3”. Further, it is explicitly noted that the following set of embodiments represents a suitably structured part of the general description directed to preferred aspects of the present invention, and, thus, suitably supports, but does not represent the claims of the present invention.
1. A process for preparing at least one polyisocyanate R(—NCO)x from CO₂, the process comprises
   (i) providing a stream S1 comprising CO₂;
   (ii) providing a stream S2 comprising H₂;
   (iii) passing and contacting a portion of S1 obtained according to (i) with a portion of S2 obtained according to (ii) into a reaction unit RURwGs for a reverse water gas shift reaction, obtaining a stream S3 comprising CO and H₂ and a stream SH comprising H₂O;
   (iv) preparing at least one polyamine R(—NH 2)x, with x=2 or more, comprising
      (iv.1) passing a portion of S3 obtained according to (iii), or a portion of S1 and a portion of S2 obtained according to (i) and (ii), respectively, into an alcohol production unit PU1 and contacting CO and H₂ comprised in said portion of S3 into PU1, or contacting S1 and S2 in PU1, obtaining a stream SM comprising an alcohol having 1 to 4 carbon atoms; or
      (iv.1′)passing a portion of S1 and a portion of S2 obtained according to (i) and (ii), respectively, into an alkane production unit PU2 and contacting said portion of S1 with said portion of S2 into PU2, obtaining a stream SM′ comprising a C₁-C₄ alkane;
      (iv.2) passing a portion of SM obtained according to (iv.1), or a portion of SM′ obtained according to (iv.1′), in an aromatic production unit PU_{MTA} and reacting the alcohol having 1 to 4 carbon atoms comprised in SM, or the C 1-C₄ alkane comprised in SM′, in PU_{MTA}, obtaining a stream SA comprising at least one aromatic compound;
      (iv.3) reacting SA obtained according to (iv.2) with nitric acid in a nitration reaction unit RU_{N}, obtaining a stream SN comprising the corresponding at least one nitrated aromatic compound;
      (iv.4)passing SN obtained according to (iv.3) into an amine production unit APU and reacting the at least one nitrated aromatic compound comprised in SN in APU, obtaining a stream S4 comprising at least one polyamine R(—NH₂)x;
   (v) preparing phosgene, comprising
      (v.1) providing a stream S5 comprising Cl₂;
      (v.2) passing a portion of S3 into a purification unit, obtaining a stream S_{CO} comprising CO and a stream S_{CH} depleted in CO compared to the portion of S3, comprising H₂;
      (v.3) passing at least a portion of S_{CO} obtained according to (v.2), into a reaction unit for preparing phosgene RU_{P} and contacting said at least portion of S_{CO}, with S5 obtained according to (v.1) into RU_{P}, obtaining a stream S6 comprising phosgene;
   (vi) passing S4 obtained according to (iv.4) comprising at least one polyamine R(—NH₂)x into a reaction unit RU and reacting S4 with phosgene comprised in S6 obtained according to (v.3) in RU, obtaining a stream SP comprising the at least one polyisocyanate R(—NCO)x.
2. The process of embodiment 1, wherein the reverse water gas shift reaction performed in RU_{RWGS} according to (iii) is performed at a temperature in the range of from 500 to 1200 °C, preferably in the range of from 700 to 1100 °C, more preferably in the range of from 800 to 1000 °C, more preferably in the range of from 850 to 1000 °C, more preferably in the range of from 900 to 1000 °C.
3. The process of embodiment 1 or 2, wherein the reverse water gas shift reaction performed in RU_{RWGS} according to (iii) is performed at a pressure in the range of from 0.5 to 20 bar(abs), preferably in the range of from 2 to 18 bar(abs), more preferably in the range of from 2 to 15 bar(abs).
4. The process of any one of embodiments 1 to 3, wherein the molar ratio of H 2 to CO₂ for the reverse water gas shift reaction performed in RURwGs according to (iii) is in the range of from 0.5:1 to 10:1, more preferably in the range of from 0.8:1 to 5:1, more preferably in the range of from 1:1 to 3:1, more preferably in the range of from 1.5:1 to 3:1, more preferably in the range of from 2:1 to 3:1.
5. The process of any one of embodiments 1 to 4, wherein (iii) comprises
   (iii.1) passing and contacting a portion of S1 obtained according to (i) with a portion of S2 obtained according to (ii) into a reaction unit RURwGs for a reverse water gas shift reaction, obtaining a stream S13 comprising CO, CO₂, H₂ and H₂O;
   (iii.2) cooling the stream S13 obtained according to (iii.1), in a condensation unit CU, obtaining a gaseous stream S3, depleted in water, compared to S13, comprising CO and H₂, and a liquid stream SH comprising H₂O.
6. The process of any one of embodiments 1 to 4, wherein (iii) comprises
   (iii.1) passing and contacting a portion of S1 obtained according to (i) with a portion of S2 obtained according to (ii) into a reaction unit R_{RWGS} for a reverse water gas shift reaction, obtaining a stream S13 comprising CO, CO₂, H₂ and H₂O;
   (iii.2′) comprises
   (iii.2′.1) cooling the stream S13 obtained according to (iii.1), in a condensation unit CU, obtaining a gaseous stream S23, depleted in water, compared to S13, comprising CO and H₂, and a liquid stream SH comprising H₂O;
   (iii.2′.2) passing S23 obtained according to (iii.2′.1) into an amine scrubbing unit for removing CO₂, obtaining a gaseous stream S3, depleted in CO₂, compared to S23, comprising CO and H₂.
7. The process of embodiment 5 or 6, wherein the condensation unit comprises a condensation mean and one or more heat exchangers.
8. The process of any one of embodiments 1 to 7, wherein the weight ratio of CO to H₂ in S3 is in the range of from 1:1 to 30:1, preferably in the range of from 2:1 to 20:1, more preferably in the range of from 5:1 to 15:1.
9. The process of any one of embodiments of 1 to 8, wherein from 25 to 100 Weight-%, of S3 consist of CO and H₂.
10. The process of any one of embodiments 1 to 9, wherein, according to (iv.1), from 75 to 95 weight-%, preferably from 85 to 93 weight-%, of S3 is passed in the alcohol production unit PU1.
11. The process of any one of embodiments 1 to 10, wherein PU1 comprises an alcohol production reactor, wherein (iv.1) comprises
   passing a portion of S3 into PU1 and contacting CO and H₂ comprised in said portion of S3 with a catalyst in the alcohol production reactor at a temperature in the range of from 150 to 500 °C, preferably in the range of from 180 to 320 °C, preferably in the range of from 200 to 300 °C, more preferably in the range of from 205 to 280 °C, obtaining a stream SM comprising an alcohol having 1 to 4 carbon atoms.
12. The process of any one of embodiments 1 to 10, wherein PU2 comprises an alkane production reactor, wherein (iv.1′) comprises
   passing a portion of S1 and a portion of S2 into PU2 and contacting CO₂ comprised in said portion of S1 and H₂ comprised in said portion of S2 in the alkane production reactor PU2 at a temperature in the range of from 250 to 600 °C, preferably in the range of from 280 to 500 °C, more preferably in the range of from 300 to 400 °C, obtaining a stream SM′ comprising a C₁-C₄ alkane.
13. The process of any one of embodiments 1 to 12, wherein (iv.2) comprises
   passing and contacting a portion of SM obtained according to (iv.1) or a portion of SM′ obtained according to (iv.1′) with a catalyst, in an aromatic production unit PU_{MTA}, obtaining a stream SA comprising at least one aromatic compound, wherein the aromatic compound is selected from the group consisting of benzene, toluene and xylene, more preferably benzene or toluene.
14. The process of any one of embodiments 1 to 13, wherein PU_{MTA} comprises at least one aromatic conversion reactor, preferably one or more of a fixed bed reactor and fluidized bed reactor.
15. The process of embodiment 14, as far embodiment 14 depends on embodiment 13,
   (iv.2.1) passing a portion of SM obtained according to (iv.1) in the at least one aromatic conversion reactor, preferably at least one methanol-to-aromatics conversion reactor;
   (iv.2.2) contacting the stream SM with a catalyst in the at least one reactor, preferably at a temperature in the range of from 250 to 750 °C, preferably in the range of from 300 to 600 °C, obtaining a stream SA1 comprising water and a mixture of aromatic compounds, the aromatic compounds being benzene, toluene and xylene;
   (iv.2.3) passing the stream SA1 obtained according to (iv.2.2) in a separation unit SU, obtaining a stream SA comprising benzene or toluene; or wherein, when (iv.1′) is performed, (iv.2) comprises
   (iv.2.1′) passing a portion of SM′ obtained according to (iv.1′) in the at least one aromatic conversion reactor, more preferably at least one methane-to-aromatics conversion reactor;
   (iv.2.2′) contacting the stream SM′ with a catalyst in the at least one reactor, more preferably at a temperature in the range of from 600 to 1000 °C, more preferably in the range of from 625 to 900 °C, more preferably in the range of from 650 to 850 °C, obtaining a stream SA1′ comprising a mixture of aromatic compounds, the aromatic compounds being benzene, toluene and xylene;
   (iv.2.3′) passing the stream SA1′ obtained according to (iv.2.2′) in a separation unit SU′, obtaining a stream SA comprising benzene or toluene.
16. The process of any one of embodiments 1 to 15, wherein (iv.3) comprises reacting SA obtained according to (iv.2) with nitric acid in a nitration reaction unit RU_{N}, obtaining a stream SN comprising the corresponding at least one nitrated aromatic compound, wherein preferably the at least one nitrated aromatic compound is selected from the group consisting of nitrobenzene and dinitrotoluene.
17. The process of any one of embodiments 1 to 16, wherein (iv.4) comprises passing SN obtained according to (iv.3) into an amine production unit APU being a hydrogenation unit and contacting the at least one nitrated aromatic compound comprised in SN with H₂ in the hydrogenation unit, obtaining a stream S4 comprising at least one corresponding aromatic polyamine R(—NH₂)x.
18. The process of embodiment 17, wherein (iv.4) comprises (iv.4.1) passing SN comprising dinitrotoluene obtained according to (iv.3) into a hydrogenation unit HU under hydrogenation conditions, obtaining a stream S4 comprising toluenediamine (TDA).
19. The process of any one of embodiments 1 to 16, wherein (iv.4) comprises
   (iv.4.1′) preparing an aqueous stream SF comprising formaldehyde;
   (iv.4.2′) passing SN obtained according to (iv.3) into a hydrogenation unit comprised in an amine production unit APU under hydrogenation conditions, obtaining a
   (iv.4.3′) bringing in contact S14 obtained according to (iv.4.2′) with SF obtained according to (iv.4.1′) in a condensation reactor R_{C} comprised in APU in presence of a catalyst, preferably an acidic catalyst, more preferably hydrochloric acid, obtaining a stream S4 comprising one or more of monomeric methylene diphenylene diisocyanate and polymethylene polyphenylene polyisocyanate.
20. The process of embodiment 19, wherein (iv.4.1′) comprises passing a portion of SM obtained according to (iv.1) in a reactor for preparing formaldehyde in presence of H₂O and O₂, obtaining an aqueous stream SF comprising formaldehyde.
21. The process of any one of embodiments 1 to 20, wherein (v.1) comprises contacting a stream comprising HCl with a stream comprising O₂ in presence of a catalyst, obtaining a stream S5 comprising Cl₂.
22. The process of any one of embodiments 1 to 21, wherein the purification unit according to (v.2) is a CO—H₂ separation cold box CB.
23. The process of any one of embodiments 1 to 22, wherein (v.3) comprises passing at least a portion of S_{CO} obtained according to (v.2), more preferably S_{CO} obtained according to (v.2), into a reaction unit for preparing phosgene RU_{P} and contacting said portion of S3 with S5 obtained according to (v.1) into RU_{P} in the presence of a catalyst, preferably activated carbon, obtaining a stream S6 comprising phosgene.
24. The process of any one of embodiments 1 to 23, wherein (vi) comprises
   (vi.1) introducing S4 obtained according to (iv.4) comprising at least one polyamine R(—NH₂)x and S6 obtained according to (v), and optionally a solvent, into a mixing device comprised in the reaction unit RU, preferably a mixing nozzle, wherein the solvent preferably is an aromatic solvent, more preferably a halogenated aromatic solvent, more preferably monochlorobenzene or dichlorobenzene, obtaining a reaction mixture;
   (vi.2) passing the reaction mixture obtained according to (vi.1) in one or more reactors comprised in RU, obtaining a stream SP comprising the at least one polyisocyanate R(—NCO)x.
25. The process of any one of embodiments 1 to 24, wherein, in SP, the at least one polyisocyanate R(—NCO)x is TDI, preferably a mixture of 2,4-TDI and 2,6-TDI.
26. The process of any one of embodiments 1 to 24, wherein in SP the at least one polyisocyanate R(—NCO)x is selected from the group consisting of monomeric methylene diphenylene diisocyanate, polymethylene polyphenylene polyisocyanate and a mixture of monomeric methylene diphenylene diisocyanate and polymethylene polyphenylene polyisocyanate.
27. The process of any one of embodiments 1 to 26, wherein at least 22.5%, preferably at least 30%, more preferably at least 50% of the carbon atoms in the at least one polyisocyanate R(—NCO)x originates from CO₂.
28. A production unit for carrying out the process according to any one of embodiments 1 to 27, the production unit comprising:
   a reaction unit R_{RWGS} for a reverse water gas shift reaction for obtaining S3 comprising CO and H₂;
   a means for introducing CO₂ in R_{RWGS};
   a means for introducing H₂ in R_{RWGS};
   a means for removing S3 from R_{RWGS};
   an alcohol production unit PU1 for obtaining SM or an alkane production unit PU2 for obtaining SM′;
   a means for passing a portion of S3 in PU1;
   a means for passing a portion of S1 in PU2;
   a means for removing SM from PU1 or a means for removing SM′ from PU2;
   an aromatic production unit PU_{MTA} for obtaining a stream SA comprising at least one aromatic compound;
   a means for passing SM or SM′ into P_{UMTA};
   a nitration reaction unit RU_{N} for obtaining a stream SN comprising at least one nitrated aromatic compound;
   a means for removing SN from RU_{N};
   an amine production unit APU for obtaining a stream S4 comprising at least one polyamine R(—NH₂)x, with x=2 or more;
   a means for passing SN into APU;
   a reaction unit RU_{P} for preparing phosgene as stream S6;
   a means for passing S5 in RU_{P};
   a purification unit, preferably a CO—H₂ separation cold box, for obtaining a stream S_{CO};
   a means for passing S_{CO} into R_{UP};
   a means for removing S_{CO} from the purification unit;
   a means for removing S_{CH} from the purification unit;
   a means for passing a portion of S3 into the purification or R_{UP};
   a means for removing S6 from R_{UP};
   a reaction unit RU for obtaining a stream SP comprising at least one corresponding polyisocyanate R(—NCO)x;
   a means for passing S4 into RU;
   a means for passing S6 into RU;
   a means for removing SP from RU.
29. The production unit of embodiment 28, wherein the amine production unit APU comprises a hydrogenation unit and optionally a condensation reaction unit.
30. The production unit of embodiment 29, wherein APU comprises a hydrogenation unit.
31. The production unit of embodiment 29, wherein APU comprises a hydrogenation unit and a condensation unit.
32. The production unit of any one of embodiments 28 to 31, further comprising a reactor for preparing formaldehyde from a portion of SM.
33. A polyisocyanate R(—NCO)x characterized in that at least at least 22.5%, preferably at least 30%, more preferably at least 50% of its carbon atoms are obtained from CO₂.
34. A polyisocyanate R(—NCO)x obtainable or obtained by a process according to any one of embodiments 1 to 27, wherein at least at least 22.5%, preferably at least 30%, more preferably at least 50% of its carbon atoms are obtained from CO₂.
35. the polyisocyanate of embodiment 33 or 34, being MDI.
36. the polyisocyanate of embodiment 33 or 34, being TDI.

In the context of the present invention, the term “gas stream” or “gaseous stream” refers to a stream in the gas or gaseous phase. Similarly, the term “liquid stream” refers to a stream in the liquid phase.

It is explicitly noted that the above set of embodiments represents a suitably structured part of the general description directed to preferred aspects of the present invention, and, thus, suitably supports, but does not represent the claims of the present invention.

In the context of the present invention, a term “X is one or more of A, B and C”, wherein X is a given feature and each of A, B and C stands for specific realization of said feature, is to be understood as disclosing that X is either A, or B, or C, or A and B, or A and C, or B and C, or A and B and C. In this regard, it is noted that the skilled person is capable of transfer to above ab-stract term to a concrete example, e.g. where X is a chemical element and A, B and C are concrete elements such as Li, Na, and K, or X is a temperature and A, B and C are concrete tem-peratures such as 10 °C, 20 °C, and 30 °C. In this regard, it is further noted that the skilled person is capable of extending the above term to less specific realizations of said feature, e.g. “X is one or more of A and B” disclosing that X is either A, or B, or A and B, or to more specific realizations of said feature, e.g. “X is one or more of A, B, C and D”, disclosing that X is either A, or B, or C, or D, or A and B, or A and C, or A and D, or B and C, or B and D, or C and D, or A and Band C, or A and B and D, or B and C and D, or A and B and C and D.

The present invention is further illustrated by the following examples.

### EXAMPLES

### Example 1

### Process for Preparing MDI From CO₂ According to the Present Invention

The process is based on the flow diagram of FIG. 3. 4451 kg/h of a purified CO₂ stream was fed to a RWGS plant and mixed with a CO₂ recycling stream of about 2150 kg/h, the two streams forming stream S1. The CO₂ was mixed with a stream S2 of 620 kg/h of hydrogen and then partially converted to CO and water in a reactor R_{RWGS} at 900 °C and a pressure of 5 bar(abs). The conversion of the CO₂ to CO was approximately 67%. The reaction mixture (stream S13) was cooled down via a condensation unit and about 1720 kg/h of water with about 1.7 wt.-% of dissolved CO₂ was condensed out. The remaining gas phase (stream S23) was fed to an amine scrubbing unit, where CO₂ (approx. 2150 kg/h) was separated, then thermally re-leased from the amine solution and fed back to the reaction as a portion of S1. A gas mixture (stream S3) of approx. 3232 kg/h remained, containing approximately 87 wt.-% of CO, 13 wt.-% of H₂ and traces of methane.

Approx. 17% of said stream S3 were fed into a CO—H₂ separation cold box, where approx. 371 kg/h of a purified CO-stream S_{CO} were yield. The remaining gas stream S_{CH} of 165 kg/h with approx. 42 weight-% H₂ and 58% CO was recycled in R_{RWGS}.

The remaining 83% of stream S3 obtained from the R_{RWGS} were fed into a methanol production unit PU1, where approx. 2971 kg/h MeOH (stream SM) were yield.

A portion of stream SM comprising methanol (2567 kg/h) was converted to light hydrocarbons (<=C₄) in a first stage in an MTA production unit and then, after separation of water and light boiling compounds in the second stage, largely converted to benzene, toluene and xylene (BTX), in said MTA production unit. In the process for obtaining BTX, an LPG fraction of about 282 kg/h, a wastewater fraction of about 1476 kg/h, and a heavy-boiling aromatics fraction of about 796 kg/h were obtained. This was further distilled for obtaining about 160 kg/h of a gaso-line-like fraction, 51 kg/h of benzene, 280 kg/h of toluene and 305 kg/h of xylene isomers.

The benzene stream was mixed with about 934 kg/h of benzene from a different source and a recycle stream of about 100 kg/h of benzene, obtaining stream SA. The stream SA was then passed through a nitration reaction unit RU_{N}, namely in an adiabatic nitration reactor, and then converted to nitrobenzene with about 20386 kg/h of nitrating acid (4.2 wt.-% of HNO₃, 64.8 wt. % of H₂SO₄ und 31 wt.-% water) at a stoichiometric benzene excess over HNO₃ of about 10%. The reaction mixture was phase separated and the diluted sulfuric acid was recycled back into the nitration reaction unit RU_{N} after concentration in a flash system. The organic phase was washed, excess benzene was separated to give about 1552 kg/h of a stream SN comprising nitrobenzene.

The portion of the methanol stream SM from the methanol production unit that was not fed to the MTA production unit (about 224 kg/h) was catalytically reacted in RF with air and water in an oxidative dehydrogenation adiabatic to about 378 kg/h of a 50 weight-% aqueous formaldehyde solution (stream SF) and used in the condensation of the aniline to MDA in APU.

The stream SN comprising nitrobenzene was then catalytically hydrogenated in the gas phase to aniline in a hydrogenation unit, namely a fluidized bed, comprised in an amine production unit APU. Water (approx. 454 kg/h) was separated and the aniline was purified by distillation to give approx. 1174 kg/h of aniline.

The aniline was mixed with a recycle-aniline-stream of about 587 kg/h and about 345 kg/h of a 30 wt.-% HCl and then partially converted to MDA in a continuous process in a reactor comprised in APU using about 378 kg/h of a 50 wt.-% aqueous formaldehyde solution (stream SF). The reaction mixture was neutralized with approx. 238 kg/h of a 50 wt.-% NaOH and separated into an aqueous and an organic phase in a subsequent separation. The organic phase was washed with water to remove remaining traces of NaOH or NaCl, and then dissolved water and excess aniline are evaporated off (source of the recycle-aniline-stream). In this way, approximately 1250 kg/h of an MDA stream S4 was obtained.

Said stream S4 was mixed with approx. 1875 kg/h monochlorobenzene (MCB) and mixed in a reaction mixing nozzle with 4460 kg/h of a phosgene-containing stream S6 (70 wt.-% phosgene, 30 wt.-% monochlorobenzene) (molar phosgene excess 150%). Thereby, the amine groups of the MDA were converted into the intermediate compounds carbamoyl chloride and amine hydrochloride. These were partly converted to isocyanates (MDI) in a reaction unit RU. In particular, they were converted in a mixing nozzle and partly (more or less completely) in a downstream reactor cascade and a final reaction column. In said reaction column comprised in RU, 920 kg/h of HCl, about 1872 kg/h of excess phosgene and some solvent (about 532 kg/h) were also discharged in gaseous form in the head of the column. In the distillation sump, about 4258 kg/h of an approximate 37wt.-% solution of MDI in MCB was obtained. In a multi-stage distillation, the solvent was removed and the MDI was thermally dechlorinated to obtain about 1578 kg/h of crude MDI at the end. The gaseous mixture at the top of the column was fed to an absorption column where phosgene is washed out with about 805 kg/h of undercooled MCB and about 920 kg/h of HCl was obtained at the top of the column. The mixture obtained in the sump (1872 kg/h phosgene and 1337 kg/h MCB) was mixed with about 1248 kg/h fresh phosgene and served then as phosgene feed in the reaction mixing nozzle of RU.

The 1248 kg/h of fresh phosgene were obtained in a heterogeneous gas phase catalysis on an activated carbon. For this purpose, the CO stream obtained in the cold-box (371 kg/h) was mixed with approx. 895 kg/h chlorine (CO excess of approx. 5%) and converted to phosgene in a tube bundle reactor (RU_{P}). The phosgene was then condensed out and fed into the reaction unit RU.

With the described process, the two carbons in the NCO groups as well as the carbon of the methyl bridge in an MDI compound were completely obtained from CO₂. Of the 12 aromatic carbon atoms, statistically 5.17% (portion of CO₂-based benzene in the total benzene amount) corresponding to 0.62 C atoms. In total, 2+1+0.62=3.62 of the 15 carbon atoms are CO₂-based, corresponding to 24.1%.

### Comparative Example 1

### Process for Preparing Polyisocyanates Not According to the Present Invention but as in WO 2021/089737

After applying the process described in WO 2021/089737 to the preparation of MDI, 2 of the 15 carbon compounds (the carbon in the NCO group) are obtained from CO₂ corresponding to a content of 13.3%.

Thus, the process according to the present invention significantly increases the proportion of CO₂-based carbon in MDI rendering the process of the present invention more sustainable than the one of the prior art.

### DESCRIPTION OF THE FIGURES

FIG. 1 is a schematic representation of a production unit used for the process according to preferred embodiments of the invention (production of TDI).

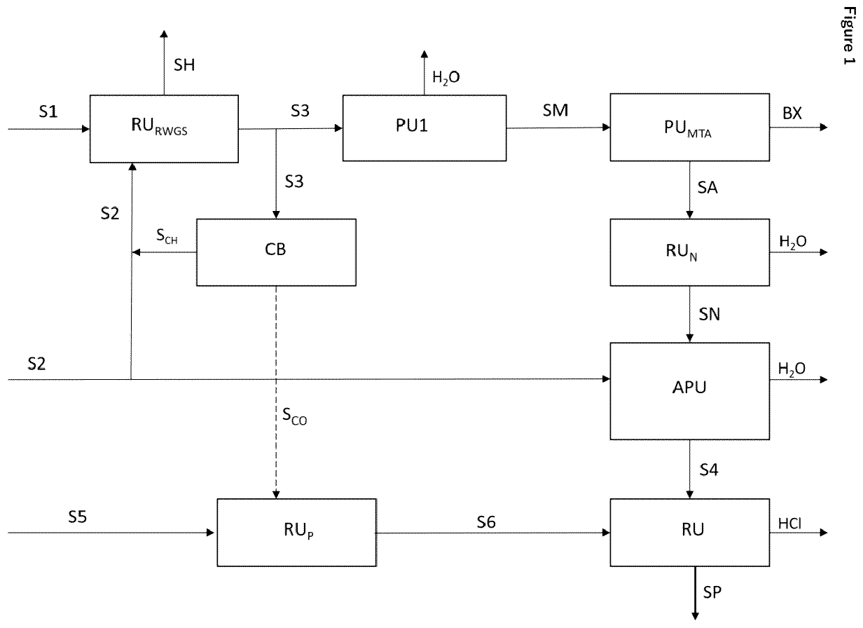

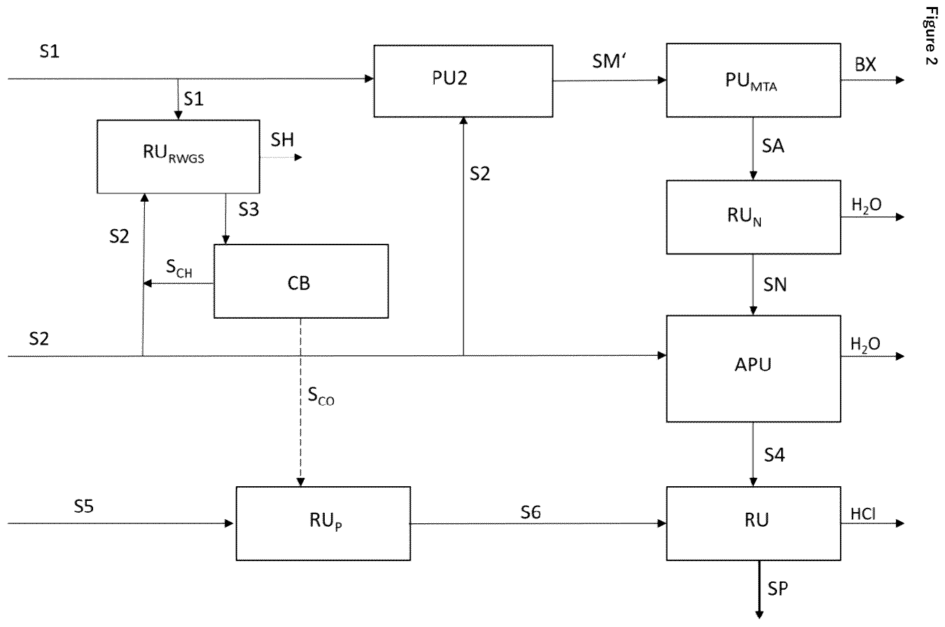

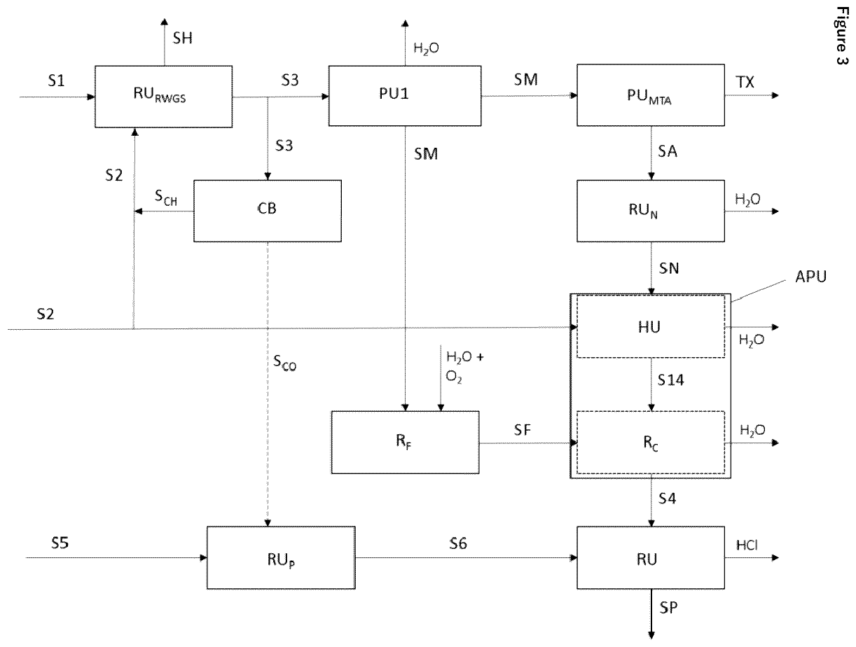

The production comprises a reaction unit RURwGs for a reverse water gas shift reaction, an alcohol production unit PU1, namely a methanol production unit, an aromatic production unit PU_{MTA}, namely a methanol to aromatics production unit, a nitration reaction unit RU_{N}, an amine production unit APU, a reaction unit RU for phosgenation, a reaction unit for preparing phosgene RU_{P} and a CO—H₂ separation cold box CB. The streams S1 (CO₂) and S2 (H₂) are introduced and reacted into RU_{RWGS} obtaining a stream S3 comprising CO and H₂ and water as stream SH. A portion of S3 is passed through PU1 obtaining a stream SM comprising methanol. The stream SM is then passed through PU_{MTA}, obtaining a stream SA comprising toluene and another stream BX comprising benzene and xylene. Said stream is not used in the process for preparing TDI but can be used in other parallel processes not illustrated in FIG. 1. Further, it is also conceivable that further demethylation of xylene takes place to produce more toluene. The stream SA is then passed in RU_{N} to obtain a stream SN comprising dinitrotoluene, said stream SN being then passed in APU for hydrogenation with a portion of S2. Water is removed from PU1, RU_{N} and APU. The stream S4 comprising toluene diamine (TDA) is removed from APU and passed through RU with S6 comprising phosgene in the optional presence of a solvent, preferably MCB. Phosgene in S6 is obtained from the reaction of S5 (Cl₂ source) and S_{CO}, a purified CO-stream removed from CB (purification unit), into RU_{P}. S_{CH} comprising H₂ is removed from CB.

The crude product TDI is removed from RU as SP.

FIG. 2 is a schematic representation of a production unit used for the process according to preferred embodiments of the invention (production of TDI).

The production comprises a reaction unit RURwGs for a reverse water gas shift reaction, an alkane production unit PU2, namely a methane production unit, an aromatic production unit PU_{MTA}, namely a methane to aromatics production unit, a nitration reaction unit RU_{N}, an amine production unit APU, a reaction unit RU for phosgenation, a reaction unit for preparing phosgene RU_{P} and a CO—H₂ separation cold box CB. A portion of S1 (CO₂) and a portion of S2 (H₂) are introduced and reacted into RURwGs obtaining a stream S3 comprising CO and H₂ and water as stream SH. A portion of S1 (CO₂) and a portion S2 (H₂) are introduced and passed through PU2 obtaining a stream SM′ comprising methane. The stream SM′ is then passed through PU_{MTA}, obtaining a stream SA comprising toluene and another stream BX comprising benzene and xylene. Said stream is not used in the process for preparing TDI but can be used in other parallel processes not illustrated in FIG. 2. Further, it is also conceivable that further demethylation of xylene takes place to produce more toluene. The stream SA is then passed through RU_{N} to obtain a stream SN comprising dinitrotoluene, said stream SN being then passed through APU for hydrogenation with a portion of S2. Water is removed from RU_{N} and APU. The stream S4 comprising toluene diamine (TDA) is removed from APU and passed through RU with S6 comprising phosgene in the optional presence of a solvent, preferably MCB. Phosgene in S6 is obtained from the reaction of S5 (Cl₂ source) and S_{CO}, a purified CO-stream removed from CB, substantially free, preferably free of H₂, into RU_{P}. S_{CH} comprising H₂ is removed from CB. The crude product TDI is removed from RU as SP.

FIG. 3 is a schematic representation of a production unit used for the process according to preferred embodiments of the invention (production of MDI−mixture of monomeric+polymeric MDI).

The production unit illustrated in FIG. 2 comprises the same elements as in the production unit of FIG. 1, except that APU comprises a hydrogenation unit HU and a condensation reactor R_{C}. Further, compared to FIG. 1, the production unit comprises a reactor RF for preparing formaldehyde. The streams S1 (CO₂) and S2 (H₂) are introduced and reacted into RURWGS obtaining a stream S3 comprising CO and H₂ and water as stream SH. A portion of S3 is passed through PU1 obtaining a stream SM comprising methanol. The stream SM is then passed through PU_{MTA}, obtaining a stream SA comprising benzene and another stream TX comprising toluene and xylene. Said stream is not used in the process for preparing MDI but can be used in other parallel processes not illustrated in FIG. 3. Further, it is also conceivable that further demethylation of xylene takes place to produce more benzene. The stream SA is then passed through RU_{N} to obtain a stream SN comprising nitrobenzene, said stream SN being then passed through HU comprised in APU for hydrogenation with a portion of S2, obtaining a stream S14 comprising aniline. The stream S14 is then reacted in R_{C} with a stream SF comprising formaldehyde in presence of a catalyst, preferably HCl, for obtaining a stream S4 comprising diaminodiphenylmethane (MDA). Water is removed from PU1, RU_{N} and APU. The stream S4 comprising MDA is removed from APU and passed through RU with S6 comprising phosgene in the presence of a solvent, preferably MCB. Phosgene in S6 is obtained from the reaction of S5 (Cl₂ source) and S_{CO},a purified CO-stream removed from CB, substantially free, preferably free of H₂, into RU_{P}. S_{CH} comprising H₂ is removed from CB. Formaldehyde in SF is obtained from a portion of SM reacted with water and oxygen in R_{F}. The crude product MDI is removed from RU as SP.

### CITED LITERATURE

WO 2021/089737 A1
WO 2020/239716 A1
E. Rezaei, S. Dzuryk “Techno-economic comparison of reverse water gas shift reaction to steam and dry methane reforming reactions for syngas production”, Chemical Engineering Research and Design, Vol 144(2019 ), S. 354-369
EP2175986
CN103183346
U.S. Pat. No. 8,946,308
DE69808983
EP3901126
EP3441381
EP0336378
EP2100869
EP2281792
EP0128400
“Ullmann's Encyclopedia of industrial chemistry”, Wiley-VCH Verlag Gmbh & Co KGaA, Weinheim, 2012 & 2016: in particular J. Ott et. al. “Methanol” (2012 & 2016); G. Booth “Nitro compounds, Aromatic” Vol. 24(2012 ); T. Kahl et. al. “Aniline” Vol. 3(2012 ); and A. W. Franz et. al. “Formaldehyde” (2016)
Sofiane Arab: “Development of a process for methanol synthesis from CO₂”. PhD, Université Nancy, 2014
Laetitia Angelo “Development of catalysts for the carbon dioxide hydrogenation into methanol”, PhD Laboratoire des matériaux, surfaces et procédés pour la catalyse, 2014
EP 0011150
U.S. Pat. No. 2,786,876
EP0090284
CN104496743
CN104549481
EP2188234
W. O. Haag, R. M. Lago, and P. G. Rodewald, J. Mol. Catal. 17, 161(1982 )
R. Le Van Mao, P. Levesque, B. Sjariel, and D. T. Nguyen, Can. J. Chem. Eng. 64,462 (1986)
G. Pop and co-workers, Ind. Eng. Chem. Prod. Res. Dev.25, 208-213 (1986)
U.S. Pat. No. 11,084,764
EP2595943
US2011060176
Ismagilov, Z. R., et al., “Direct Conversion of Methane on Mo/ZSM-5 Catalysts to Produce Benzene and Hydrogen: Achievements and Perspectives,” Energy & Environmental Science 1, 5(2008 ), 526-541
H. Hermann et al, “Industrial Nitration of Toluene to Dinitrotoluene”, in L.F. Albright et al, eds, Nitration: Recent Laboratory and Industrial Developments, ACS Symposium Series 623, Chapter 21 (Washington: American Chemical Society, 1996)
WO 2007/134771 A1
WO 2011/111351 A1
WO 2013/004651 A1
WO 2013/060628 A1
U.S. Pat. No. 2,418,930
WO 2012/025483 A1
WO 2007137685
U.S. Pat. No. 6,022,634
WO2003/31690
WO1999031297
EP1103636
U.S. Pat. No. 3,236,760
EP0004903
Ullmann's Encyclopedia of industrial chemistry, Chapter “Phosgene” 5^{th} Ed., Vol. A 19, p 413 ff., VCH Verlagsgesellschaft mbH, Weinheim, 1991
C. Ryan et. al. “Phosgene and related carbonyl halides”, Elsevier Science, 1996, ISBN 978-0-08-053880-8

1-18. (canceled)

19. A process for preparing at least one polyisocyanate R(—NCO)x from CO₂, the process comprising:
(i) providing a stream S1 comprising CO₂;
(ii) providing a stream S2 comprising H₂;
(iii) passing and contacting a portion of S1 obtained according to (i) with a portion of S2 obtained according to (ii) into a reaction unit RU_{RWGS} for a reverse water gas shift reaction, obtaining a stream S3 comprising CO and H₂ and a stream SH comprising H₂O;
(iv) preparing at least one polyamine R(—NH 2)x, with x=2 or more, comprising:
(iv.1) passing a portion of S3 obtained according to (iii), or a portion of S1 and a portion of S2 obtained according to (i) and (ii), respectively, into an alcohol production unit PU1 and contacting CO and H₂ comprised in said portion of S3 into PU1, or contacting S1 and S2 in PU1, obtaining a stream SM comprising an alcohol having 1 to 4 carbon atoms; or
(iv.1′) passing a portion of S1 and a portion of S2 obtained according to (i) and (ii), respectively, into an alkane production unit PU2 and contacting said portion of S1 with said portion of S2 into PU2, obtaining a stream SM′ comprising a C₁-C₄ alkane;
(iv.2) passing a portion of SM obtained according to (iv.1), or a portion of SM′ obtained according to (iv.1′), in an aromatic production unit PU_{MTA} and reacting the alcohol having 1 to 4 carbon atoms comprised in SM, or the C 1-C₄ alkane comprised in SM′, in PU_{MTA}, obtaining a stream SA comprising at least one aromatic compound;
(iv.3) reacting SA obtained according to (iv.2) with nitric acid in a nitration reaction unit RU_{N}, obtaining a stream SN comprising the corresponding at least one nitrated aromatic compound; and
(iv.4) passing SN obtained according to (iv.3) into an amine production unit APU and reacting the at least one nitrated aromatic compound comprised in SN in APU, obtaining a stream S4 comprising at least one polyamine R(—NH₂)x;
(v) preparing phosgene, comprising:
(v.1) providing a stream S5 comprising Cl₂;
(v.2) passing a portion of S3 into a purification unit, obtaining a stream S_{CO} comprising CO and a stream S_{CH} depleted in CO compared to the portion of S3, comprising H₂; and
(v.3) passing at least a portion of S_{CO} obtained according to (v.2), into a reaction unit for preparing phosgene RU_{P} and contacting said at least portion of S_{CO}, with S5 obtained according to (v.1) into RU_{P}, obtaining a stream S6 comprising phosgene; and
(vi) passing S4 obtained according to (iv.4) comprising at least one polyamine R(—NH₂)x into a reaction unit RU and reacting S4 with phosgene comprised in S6 obtained according to (v.3) in RU, obtaining a stream SP comprising the at least one polyisocyanate R(—NCO)x.

20. The process of claim 19, wherein the reverse water gas shift reaction performed in RU_{RWGS} according to (iii) is performed at a temperature in the range of from 500 to 1200 °C

21. The process of claim 19, wherein the reverse water gas shift reaction performed in RU_{RWGS} according to (iii) is performed at a pressure in the range of from 0.5 to 20 bar(abs).

22. The process of claim 19, wherein (iii) comprises:
(iii.1) passing and contacting a portion of S1 obtained according to (i) with a portion of S2 obtained according to (ii) into a reaction unit RU_{RWGS} for a reverse water gas shift reaction, obtaining a stream S13 comprising CO, CO₂, H₂ and H₂O;
(iii.2) cooling the stream S13 obtained according to (iii.1), in a condensation unit CU, obtaining a gaseous stream S3, depleted in water, compared to S13, comprising CO and H₂, and a liquid stream SH comprising H₂O; or (iii.2′) comprises:
(iii.2′.1) cooling the stream S13 obtained according to (iii.1), in a condensation unit CU, obtaining a gaseous stream S23, depleted in water, compared to S13, comprising CO and H₂, and a liquid stream SH comprising H₂O;
(iii.2′.2) passing S23 obtained according to (iii.2′.1) into an amine scrubbing unit for removing CO₂, obtaining a gaseous stream S3, depleted in CO₂, compared to S23, comprising CO and H₂.

23. The process of claim 19, wherein PU1 comprises an alcohol production reactor, wherein (iv.1) comprises passing a portion of S3 into PU1 and contacting CO and H₂ comprised in said portion of S3 with a catalyst in the alcohol production reactor at a temperature in the range of from 150 to 500 °C, obtaining a stream SM comprising an alcohol having 1 to 4 carbon atoms.

24. The process of claim 19, wherein PU2 comprises an alkane production reactor, wherein (iv.1′) comprises passing a portion of S1 and a portion of S2 into PU2 and contacting CO₂ comprised in said portion of S1 and H₂ comprised in said portion of S2 in the alkane production reactor PU2 at a temperature in the range of from 250 to 600 °C, obtaining a stream SM′ comprising a C₁-C₄ alkane.

25. The process of claim 19, wherein (iv.2) comprises passing and contacting a portion of SM obtained according to (iv.1) or a portion of SM′ obtained according to (iv.1′), with a catalyst in an aromatic production unit PU_{MTA}, obtaining a stream SA comprising at least one aromatic compound, wherein the aromatic compound is selected from the group consisting of benzene, toluene and xylene.

26. The process of claim 25, wherein, when (iv.1) is performed, (iv.2) comprises:
(iv.2.1) passing a portion of SM obtained according to (iv.1) in at least one aromatic conversion reactor comprised in PU_{MTA};
(iv.2.2) contacting the stream SM with a catalyst in the at least one reactor at a temperature in the range of from 250 to 750 °C obtaining a stream SA1 comprising water and a mixture of aromatic compounds, the aromatic compounds being benzene, toluene and xylene; and
(iv.2.3) passing the stream SA1 obtained according to (iv.2.2) in a separation unit SU, obtaining a stream SA comprising benzene or toluene; or
wherein, when (iv.1′) is performed, (iv.2) comprises
(iv.2.1′) passing a portion of SM′ obtained according to (iv.1′) in at least one aromatic conversion reactor;
(iv.2.2′) contacting the stream SM′ with a catalyst in the at least one reactor at a temperature in the range of from 600 to 1000 °C obtaining a stream SA 1′ comprising a mixture of aromatic compounds, the aromatic compounds being benzene, toluene and xylene; and
(iv.2.3′) passing the stream SA1′ obtained according to (iv.2.2′) in a separation unit SU′, obtaining a stream SA comprising benzene or toluene.

27. The process of claim 19, wherein (iv.3) comprises reacting SA obtained according to (iv.2) with nitric acid in a nitration reaction unit RU_{N}, obtaining a stream SN comprising the corresponding at least one nitrated aromatic compound, wherein the at least one nitrated aromatic compound is selected from the group consisting of nitrobenzene and dinitrotoluene.

28. The process of claim 19, wherein (iv.4) comprises passing SN obtained according to (iv.3) into an amine production unit APU being a hydrogenation unit and contacting the at least one nitrated aromatic compound comprised in SN with H₂ into the hydrogenation unit, obtaining a stream S4 comprising at least one corresponding aromatic polyamine R(—NH₂)x.

29. The process of claim 28, wherein (iv.4) comprises:
(iv.4.1) passing SN comprising dinitrotoluene obtained according to (iv.3) into a hydrogenation unit HU under hydrogenation conditions, obtaining a stream S4 comprising toluenediamine (TDA).

30. The process of claim 19, wherein (iv.4) comprises:
(iv.4.1′) preparing an aqueous stream SF comprising formaldehyde;
(iv.4.2′) passing SN obtained according to (iv.3) into a hydrogenation unit comprised in an amine production unit APU under hydrogenation conditions, obtaining a stream S14 comprising aniline; and
(iv.4.3′) bringing in contact S14 obtained according to (iv.4.2′) with SF obtained according to (iv.4.1′) in a condensation reactor R_{C} comprised in APU in presence of a catalyst, obtaining a stream S4 comprising one or more of monomeric methylene diphenylene diisocyanate and polymethylene polyphenylene polyisocyanate.

31. The process of claim 30, wherein (iv.4.1′) comprises passing a portion of SM obtained according to (iv.1) in a reactor for preparing formaldehyde in presence of H₂O and O₂, obtaining an aqueous stream SF comprising formaldehyde.

32. The process of claim 19, wherein the purification unit according to (v.2) is a CO—H₂ separation cold box CB.

33. The process of claim 19, wherein (v.3) comprises passing at least a portion of Sco obtained according to (v.2) into a reaction unit for preparing phosgene RU_{P} and contacting said portion of S3 with S5 obtained according to (v.1) into RU_{P} in the presence of a catalyst, obtaining a stream S6 comprising phosgene.

34. The process of claim 19, wherein (vi) comprises:
(vi.1) introducing S4 obtained according to (iv.4) comprising at least one polyamine R(—NH₂)x and S6 obtained according to (v), and optionally a solvent, into a mixing device comprised in the reaction unit RU, wherein the solvent is an aromatic solvent, obtaining a reaction mixture; and
(vi.2) passing the reaction mixture obtained according to (vi.1) in one or more reactors comprised in RU, obtaining a stream SP comprising the at least one polyisocyanate R(—NCO)x.

35. A production unit for carrying out the process according to claim 19, the production unit comprising:
a reaction unit R_{RWGS} for a reverse water gas shift reaction for obtaining S3 comprising CO and H₂;
a means for introducing CO₂ in R_{RWGS};
a means for introducing H₂ in R_{RWGS};
a means for removing S3 from R_{RWGS};
an alcohol production unit PU1 for obtaining SM or an alkane production unit PU2 for obtaining SM═;
a means for passing a portion of S3 in PU1;
a means for passing a portion of S1 in PU2;
a means for removing SM from PU1 or a means for removing SM′ from PU2;
an aromatic production unit PU_{MTA} for obtaining a stream SA comprising at least one aromatic compound;
a means for passing SM or SM′ into PU_{MTA};
a nitration reaction unit RU_{N} for obtaining a stream SN comprising at least one nitrated aromatic compound;
a means for removing SN from RU_{N};
an amine production unit APU for obtaining a stream S4 comprising at least one polyamine R(—NH₂)x, with x=2 or more;
a means for passing SN into APU;
a reaction unit RU_{P} for preparing phosgene as stream S6;
a means for passing S5 in RU_{P};
a purification unit for obtaining a stream S_{CO};
a means for passing S_{CO} into RU_{P};
a means for removing S_{CO} from the purification unit;
a means for removing S_{CH} from the purification unit;
a means for passing a portion of S3 into the purification or RU_{P};
a means for removing S6 from RU_{P};
a reaction unit RU for obtaining a stream SP comprising at least one corresponding polyisocyanate R(—NCO)x;
a means for passing S4 into RU;
a means for passing S6 into RU; and
a means for removing SP from RU.

36. A polyisocyanate R(—NCO)x characterized in that at least at least 22.5% of its carbon atoms are obtained from CO₂; the polyisocyanate R(—NCO)x being obtainable or obtained by a process according to claim 19.
